(19) United States
(12) Patent Application Publication
(10) Pub. No.: US 2002/0039556 A1
(43) Pub. Date: Apr. 4, 2002

(54) Stabilised phospholipid compositions

(76) Inventors: Kari Dyvik, Oslo (NO); Harald Dugstad, Oslo (NO); Jo Klaveness, Oslo (NO); Roald Skurtveit, Oslo (NO); Jonny Ostensen, Oslo (NO); Kiyoto Yachi, Tokyo (JP)

Correspondence Address: Richard E. Fichter, BACON & THOMAS, PLLC, Fourth Floor, 625 Slaters Lane, Alexandria, VA, 22314-1176, US

(21) Appl. No.: 09/982,196

(22) Filed: Oct. 19, 2001

Related U.S. Application Data
(63) Continuation of application No. 09/043,575, filed on Jul. 13, 1998.
Application No. 09/043,575 is a 371 of international application No. PCT/GB96/02364, filed on Sep. 25, 1996.

(30) Foreign Application Priority Data
Sep. 27, 1995 (GB) 9519654.9

(51) Int. Cl. A61K 51/00; A61K 9/127; A61K 49/04

(52) U.S. Cl. 424/1.21; 424/9.321; 424/9.51; 424/450

(57) ABSTRACT

The stability of phospholipid compositions is enhanced by the inclusion of a buffer system comprising ammonia or a water soluble amine having a pH at 15° C. of less than or equal to 9.5.

This invention relates to stabilised aqueous phospholipid compositions.

Phospholipid compositions are used in a variety of diagnostic, therapeutic and cosmetic applications. For example, lipid compositions, in particular liposomes, are used to incorporate diagnostic and therapeutic agents, as vehicles for transfer of genetic material, as immunological adjuvants, in preparation of vaccines and in cancer detection. Clearly, the stability of the phospholipid is important for the protection of any entrapped substance from degradation reactions and also for optimum performance of the phospholipid itself.

One particular area of interest is in the field of diagnostic imaging. Contrast agents are employed to effect imaging enhancement in a variety of diagnostic techniques, the most important of these being X-ray imaging, magnetic resonance imaging (MRI), ultrasound imaging and nuclear medicine imaging. There is a continuing need for contrast agents which combine good storage stability and stability in vivo. Another area of particular interest is the development of stable phospholipid compositions for use in techniques involving autoclavation.

Studies have shown the use of trometamol and related buffer compounds to have a specific effect on the hydrolysis of phospholipids. Many of these studies show general acid/base catalysis by the trometamol buffer with increased hydrolysis of the phospholipids with increasing concentration of buffer species (see for example Journal of Pharmaceutical Sciences 82: 362-366 (1993)).

However, inhibition of the hydrolysis of phospholipid compositions due to an interaction between trometamol and phospholipase contained within the compositions has also been reported (see for example Biochemical and Biophysical Res. Comm. 84: 238-247 (1978); Biochem. J. 203: 799-801 (1982); Archives of Insect Biochemistry and Physiology 14: 1-12 (1990); and Journal of Bacteriology 175: 4298-4306 (1993)).

In Chemistry and Physics of Lipids 60: 93-99 (1991), the stability of phospholipids in liposomal aqueous suspension against oxidative degradation in air was investigated. It was demonstrated that lecithin was more resistant to hydrolysis in trometamol buffer than in pure water. This was indicated to relate specifically to the naturally occurring phospholipid having polyunsaturated fatty acid chains which are readily susceptible to peroxidation by a free-radical mechanism. As indicated in the disclosure, it is well-known that ultrasonic irradiation of water promotes the production of hydroxyl free radicals and hydrogen peroxide and that these active oxygen species are involved in oxidative degradation of phospholipids. The trometamol buffer appeared to provide resistance to hydrolysis and the reference indicates that the reduced oxidation/ hydrolysis observed is due to the fact that trometamol acts as an efficient scavenger of hydroxyl free radicals. A similar conclusion is reached in J.Pharm. Pharmacol. 45: 490-495 (1993), where a protective effect of buffers such as trometamol against lipid peroxidation is reported.

In WO-95/26205 (Nycomed/Daiichi) there are described diagnostic compositions containing multilamellar liposomes containing at least one imaging agent and being suspended in an aqueous medium containing said imaging agent, wherein the liposomes comprise a neutral phospholipid and a charged phospholipid, the average particle diameter of the liposomes is 50-3000 nm and the concentration of imaging agent in any aqueous phase filling the interior of the liposomes is substantially the same as that in the aqueous medium in which the liposomes are suspended.

It has now, surprisingly, been found that substantially saturated phospholipid compounds can be stabilised by buffers, the buffers providing a reduced degree of aqueous hydrolysis of the phospholipids.

Thus, viewed from one aspect the present invention provides an aqueous lipid composition, preferably a liposomal composition and preferably a composition in physiologically tolerable form, comprising one or more substantially saturated phospholipids in combination with a buffer system comprising ammonia or a water soluble amine having a pH at 15° C. of less than or equal to 9.5, with the proviso that where said phospholipids comprise a combination of charged and neutral phospholipids and said composition is a liposomal composition containing a nonionic multiply hydroxylated X-ray contrast agent then said agent is not present within the liposomes and within the surrounding aqueous medium at substantially the same concentration.

The diagnostic compositions disclosed in WO-95/26205 thus are specifically disclaimed.

According to a further aspect of the present invention we provide a method for stabilising a substantially saturated phospholipid composition, which method comprises including in a substantially saturated phospholipid composition a buffer system comprising ammonia or a water soluble amine having a pH at 15° C. of less than or equal to 9.5, other than by adding a said buffer system to a liposomal composition containing a nonionic multiply hydroxylated X-ray contrast agent having said agent present within the liposomes and within the surrounding aqueous medium at substantially the same concentration.

For liposomal compositions, the buffer may be added before or after liposome generation.

Viewed from a further aspect the invention also provides a method of contrast enhanced imaging in which a contrast medium is administered to a subject (eg. a human or non-human animal, preferably a mammal) and an image of the subject is generated, characterised in that as said contrast medium is used a composition according to the invention containing a contrast effective material. If desired, the contrast medium may be administered after activation of the contrast effective material, eg. by hyperpolarization.

Viewed from a still further aspect the invention provides a method of treatment in which a therapeutic or prophylactic agent is administered to a subject (eg. a human or non-human animal, preferably a mammal), characterised in that there is administered a composition according to the invention containing a said therapeutic or prophylactic agent.

Viewed from a yet further aspect the invention provides a method of cosmetic treatment in which a cosmetic agent is administered to a subject (eg. a human or non-human animal, preferably a mammal) characterised in that there is administered a composition according to the invention containing a said cosmetic agent.

In these methods, the compositions administered should contain an effective amount of the active agent (the contrast effective material, the therapeutic or prophylactic agent or the cosmetic agent), namely an amount sufficient to achieve contrast enhancement or to achieve the desired therapeutic, prophylactic or cosmetic effect.

The phospholipids used in the compositions and methods of the invention may be charged or neutral (ie. carry no net charge). The use of neutral phospholipids however is particularly preferred as their protection against hydrolysis by the buffer system is particularly pronounced. Especially preferably the phospholipids in the compositions of the invention are entirely or substantially entirely neutral phospholipids.

The buffer systems for use in the methods or compositions of the present invention preferably have a pH of 6.0 to 9.5 at room temperature (15° C.), more preferably 6.5 to 8.0, particularly preferably 6.8 to 7.8.

The compositions of the present invention show a reduced degree of hydrolysis of the phospholipid(s) when compared with formulations not including the specified buffer system. Preferred compositions according to the present invention show a greater than 5% reduction in the extent of hydrolysis over a given time (eg. a normal shelf life, for example 30 days or more) than occurs with formulations not including the buffer; more preferred compositions show a greater than 10% and most preferably greater than 25% reduction.

The stabilisation achieved is of especial advantage during storage, during processing and during exposure of the phospholipid compositions to temperature, including during autoclaving.

In a preferred embodiment of the present invention the phospholipid compositions are stable at temperatures in the range from 4 to 30° C.; in a more preferred embodiment the compositions are stable for temperatures in the range from 4 to 50° C.; in another more preferred embodiment the compositions are stable for temperatures in the range of 4 to 125° C. (which includes autoclaving).

The phospholipid compositions are preferably stable under storage for a period of up to 2 years, more preferably up to 3 years, particularly preferably up to 5 years. “Stable” in this context means that at least 75%, preferably at least 80%, more preferably at least 90%, of undegraded phospholipid is present in the composition after the specified storage period.

As indicated above, one particular advantage of the stabilization method of the invention is that the resulting phospholipid compositions have the ability to withstand a wide temperature range for a short period. It is preferred, therefore, that for stabilising phospholipid compositions to be autoclaved the buffer system is added prior to autoclaving.

Buffers which may be employed in the methods or compositions of the present invention are preferably those of formula (I)

NR¹R²R³ (I)

wherein R¹, R² and R³, which may be the same or different, each represents a hydrogen atom, a sugar residue, an alkyl group with 1 to 6 carbon atoms (which may carry one or more hydroxy, mercapto, carboxyl, sulphonic acid, carboxamido, imidazolyl, indolyl or hydroxy substituted phenyl groups), an alkylthio group with 1 to 6 carbon atoms and/or a group of the formula NR⁴R⁵ (in which R⁴ and R⁵, which may be the same or different, each represents a hydrogen atom, a carboxamido or —C(═NH)NH₂ group or an alkyl group with 1 to 6 carbon atoms); or any two of R¹, R² and R³ may, together with the intervening nitrogen atom, represent a pyrrolidine, morpholine or piperidine ring which may carry hydroxy, carboxyl, sulphonic acid or carboxamido groups.

Thus, for example, water soluble amines which may be employed as buffers include amino alcohols and amino sugars. More preferred amines include trometamol (tris(hydroxymethyl)methylamine, also denoted TRIS), N,N-bis(2-hydroxyethyl)-tris(hydroxymethyl)methylamine (denoted BIS-TRIS), 2-amino-2-methylpropane-1,3-diol (denoted AMPD), TES, 2-[4-(2-hydroxyethyl)-1-piperazinyl]ethanesulphonic acid (denoted HEPES), diethanolamine, meglumine, triethanolamine and ammonia.

Especially preferred amines for use according to the invention are TRIS, BIS-TRIS, TES and meglumine in view of their advantageous physiological acceptability and/or advantageous pH values at room temperature.

The phospholipids for inclusion in the compositions of the present invention are, as indicated above, comprised of substantially saturated phospholipids. The term “substantially saturated” means that the fatty acid residues of the phospholipids are fully saturated (i.e. contain no C-C double bonds) or that the extent of their unsaturation is very low, e.g. as shown by an iodine value of no more than 10, preferably no more than 5. A small proportion of unsaturated phospholipids giving an analogous overall extent of unsaturation may also be present in the compositions of the present invention. The phospholipids may be charged or neutral and may be of natural, synthetic or semi-synthetic origin (including chemically modified substantially saturated phospholipids). As mentioned above the use of neutral phospholipids is preferred.

The number of carbon atoms in the fatty acid residues is usually at least 14, preferably at least 16. The number of carbon atoms in the fatty acid residue is also preferably 26 or less, eg. 25 or less, preferably 24 or less.

Neutral phospholipids useful in the present invention include, for example, neutral glycerophospholipids, for example a fully hydrogenated naturally occurring (e.g. soybean- or egg yolk-derived) or synthetic phosphatidylcholine, particularly semisynthetic dipalmitoyl phosphatidylcholine (DPPC) or distearoyl phosphatidylcholine (DSPC), phosphatidylethanolamine (PE) or phosphatidylethanolamine-polyethyleneglycol (PE-PEG). More than one neutral phospholipid may be used.

Charged phospholipids useful in the present invention include, for example, positively or negatively charged glycerophospholipids. Negatively charged phospholipids include, for example, phosphatidylserine, for example a fully hydrogenated naturally occurring (e.g. soybean- or egg yolk-derived) or semi-synthetic phosphatidylserine, particularly semi-synthetic dipalmitoyl phosphatidylserine (DPPS) or distearoyl phosphatidylserine (DSPS); phosphatidylglycerol (PG), for example fully hydrogenated naturally occurring (e.g. soybean- or egg yolk-derived) or semi-synthetic phosphatidylglycerol, particularly semi-synthetic or synthetic dipalmitoyl phosphatidylglycerol (DPPG); or distearoyl phosphatidylglycerol (DSPG); phosphatidylinositol, for example a fully hydrogenated naturally occurring (e.g. soybean- or egg yolk-derived) or semi-synthetic phosphatidylinositol, particularly semi-synthetic or synthetic dipalmitoyl phosphatidylinositol (DPPI) or distearoyl phosphatidylinositol (DSPI); phosphatidic acid, for example a fully hydrogenated naturally occurring (e.g. soybean- or egg yolk-derived) or semi-synthetic phosphatidic acid, particularly semi-synthetic or synthetic dipalmitoyl phosphatidic acid (DPPA) or distearoyl phosphatidic acid (DSPA). Positively charged phospholipids include, for example, an ester of phosphatidic acid with an aminoalcohol, such as an ester of dipalmitoyl phosphatidic acid or distearoyl phosphatidic acid with hydroxyethylenediamine. Although such charged phospholipids are commonly used alone, more than one charged phospholipid may be used.

The concentration of the buffer for use in the method of the present invention or in the compositions of the present invention is preferably in the range 2 mM to 200 mM, more preferably 2 mM to 100 mM and particularly preferably 2 mM to 20 mM.

The molar ratio of buffer:lipid in the compositions of the present invention is preferably in the range 1:60 to 2000:1 (eg. 1:60 to 100:1), more preferably 1:60 to 1:0.02, particularly preferably 1:60 to 1:0.1. Another preferred range of molar ratio of buffer:lipid for some diagnostic and other medical applications is 1:50 to 1:0.1, more preferably 1:20 to 1:0.5, particularly preferably 1:5 to 1:1.

The concentration of phospholipids in the compositions of the present invention for imaging and medical applications is preferably in the range 0.01 mM to 120 mM, eg. 1 mM to 120 mM.

The phospholipid compositions of the present invention may be in any of the formulation types generally encountered, for example liposomes, emulsions, micelles, microemulsions, lipid particles, lipid solutions and microbubbles. They can be produced by conventional procedures for each particular formulation type.

The method and the phospholipid compositions of the present invention are suitable for use in a variety of applications and in particular those where increased stability is of especial importance. The stabilised compositions can be used in a variety of diagnostic, therapeutic and cosmetic applications and particular mention can be made of phospholipid compositions for use with contrast media (X-ray, MRI, US and scintigraphy), and for use in cancer therapy, chemotherapy, therapy for fungal infections and treatment of psoriasis.

For use as diagnostic imaging contrast media, the liposomal compositions of the invention will include a contrast-effective material, eg. in the inner cavity of the liposomes, attached to the inner or outer wall of the liposome membrane or contained within the membrane, or in the liquid medium in which the liposomes are dispersed. By contrast effective it is meant that the material is capable of enhancing contrast in the imaging modality of interest. For conventional imaging modalities, eg. X-ray, MR, ultrasound, magnetotomography, electrical impedance tomography, scintigraphy, SPECT, PET, etc. the nature of appropriate contrast effective materials is well known, for example gases (eg. air, xenon, fluorinated compounds, etc.), radiation emitters, paramagnetic, superparamagnetic, ferrimagnetic and ferromagnetic materials (eg. paramagnetic chelates of transition metals or lanthanides), heavy atom (eg. atomic number 47 or higher) compounds, eg. iodinated compounds (eg. triiodophenyl compounds). Where the contrast effective material is gaseous (at ambient or body temperature), eg. air, xenon, helium, argon, hydrogen, nitrous oxide, oxygen, nitrogen, carbon dioxide, sulphur hexafluoride, methane, acetylene, fluorinated low molecular weight (eg. C₁ to C₇) hydrocarbons (preferably perfluorocarbons such as C₂F₆, C₃F₈, C₄F₁₀ and C₅F₁₂), ¹⁹F-containing gases, etc. it is conveniently contained within the liposome membrane. Where the contrast effective material is water soluble (eg. a soluble triiodophenyl compound or a paramagnetic metal chelate) it is preferably in solution in the liposome core and especially preferably also in solution in the suspension medium.

Therapeutic or cosmetic agents may be similarly dispersed within the liposomal core, in or on the liposome membrane and/or in the suspension medium. Conventional therapeutic or cosmetic agents capable of liposomal delivery may be used.

For diagnostic compositions, e.g for X-ray and MRI/nuclear medicine, the concentration of total lipid is generally 5 mg/ml to 100 mg/ml (eg. 20 to 100 mg/ml, conveniently at least 40 or 50 mg/ml), preferably 10 mg/ml to 90 mg/ml, and more preferably 10 mg/ml to 80 mg/ml, in order to enhance encapsulation of contrast agent in the lipid. However, for ultrasound diagnostic compositions a preferred range for the concentration of total lipid is generally 0.01 mg/ml to 20 mg/ml, preferably 0.01 mg/ml to 10 mg/ml (eg. 0.5 mg/ml to 10 mg/ml).

Where agents are encapsulated in the phospholipid (particularly in liposomes) this is preferably in the form of an isotonic solution or suspension (relative to physiological osmotic pressure in the body). To obtain an isotonic solution or suspension, the agent is generally dissolved or suspended in a medium at a concentration which provides an isotonic solution. In the case where the agent alone cannot provide an isotonic solution because, for example, the solubility of the agent is insufficient, other conventional tonicity adjusters (e.g non-toxic water soluble substances) may be added to the medium so that an isotonic solution is formed. Examples of such substances include: salts such as sodium chloride; sugars such as mannitol, glucose, sucrose, mannose, galactose, sorbitol or the like; and polyhydric alcohols such as propylene glycol, glycerine and the like. If sorbitol is employed this is preferably at a concentration of 1 to 500 g/l, more preferably 0.1 to 20 g/100 ml. If glycerine is employed this is preferably at a concentration of 0.05 to 10 g/100 ml. If the phospholipid compositions are liposomal compositions, the amount of salts used is preferably as small as possible to facilitate stability of the liposomes during storage and autoclaving.

Isotonic solutions provided by means of the substances mentioned above are also preferably included in those phospholipid compositions according to the present invention which do not incorporate diagnostic, therapeutic or cosmetic agents.

The present phospholipid compositions may also contain various optional components in addition to the above-mentioned components. For example, vitamin E (α-tocopherol) and/or vitamin E acetate ester as an antioxidant may be added in an amount of 0.01 to 2 molar %, preferably 0.1 to 1 molar % relative to total amount of lipids.

Diagnostic, therapeutic and cosmetic agents referred to above may be incorporated into the phospholipid compositions of the present invention by techniques well known in the art.

As indicated above, the prior art describes the inhibition of phospholipid hydrolysis to be by an indirect mechanism involving inhibition of a phospholipase; such a mechanism clearly does not apply in the present invention since the compositions concerned do not contain phospholipase. Similarly, the reduced oxidation/hydrolysis observed in the prior art using unsaturated phospholipids cannot be important in the method of the present invention involving saturated phospholipids (not withstanding that traces of unsaturated phospholipids can be present). The method of the present invention appears to demonstrate a different inhibition mechanism involving general inhibition of the acid/base catalysed hydrolysis of phospholipid esters.

The precise mechanism involved in the method of the present invention is not fully understood, particularly in terms of the improved stability on storage. However, it is well known (see for example Journal of Pharmaceutical Sciences 82: 362-366 (1993) and Phospholipid Handbook (Marcel Dekker Inc., 1993), pages 323-324) that phospholipids can be hydrolysed to form free fatty acids and lysophospholipids, which can be further hydrolysed to the corresponding glycerophospho compounds and free fatty acids; the final hydrolysis step gives glycerophosphoric acid by hydrolysis of the phosphate head group. Hydrolysis of the ester bond between glycerol and phosphoric acid seems to be difficult since no free phosphoric acid and glycerol is detected. Evidently the use of a buffer such as trometamol inhibits the acid/base catalysed hydrolysis of the fatty ester groups, as evidenced by the reduced liberation of free fatty acids; the final hydrolysis step may also be inhibited. However, other hydrolysis mechanisms may also be involved.

The following non-limiting Examples serve to further illustrate the methods and compositions of the present invention.

### EXAMPLE 1

Composition: 1 ml containing:

| | |
|---|---|
| Hydrogenated egg phosphatidylcholine | 32 mg |
| 1,2-Dipalmitoyl-sn-glycero-3-phosphoethanolamine | 4 mg |
| 1,2-Dipalmitoyl-sn-glycero-3-phosphatidic | 4 mg |
| acid sodium | |
| Sorbitol | 50 mg |
| (Trometamol | 1 mg) |
| Water for injection ad | 1 ml |

The composition was prepared by mixing the lipids with a mixture of chloroform, methanol and water (volume ratio 80:20:0.05). The mixture was heated on a water bath (at 50° C.) to dissolve the lipids and the solvents were then removed by heating the solution in a rotary evaporator (at 50° C.). Liposomes were then prepared by homogenisation and extrusion using standard techniques (including the addition of sorbitol). The dispersion was then split into two parts and a buffer of trometamol/HCl having a pH of 7.4 was added to one of the two parts. The resulting composition was then filled into vials and autoclaved. Samples were stored at each of 30°, 40° and 50° C. for one month. The content of free fatty acids was measured before autoclaving, after autoclaving and after storage.

The presence of free fatty acids (FFA) in the phospholipid compositions was examined by elution on a thin layer chromatographic (TLC) plate coated with an 0.25 mm thick layer of silica gel 60 using a mixture of methanol, chloroform and ammonia (120:70:8 by volume) as the mobile phase. The sample was diluted 1:10 with methanol:dichloromethane (2:1 by volume) and an aliquot of the diluted sample was applied to the chromatographic plate. The amount of FFA was semi-quantified by comparison of the intensity of the spot obtained from the sample and spots from palmitic acid standards corresponding to 1.25 mg/ml to 25 mg/ml concentrations. The spots were developed with cupric sulphate spray reagent for about one hour at 170° C.

Degradation of phospholipids, measured as free fatty acids (mg/ml):

| | Without trometamol | With trometamol |
|---|---|---|
| 1 month at 30° C. | ≦5.0 | ≦2.5 |
| 1 month at 40° C. | ≦12.5 | ≦5.0 |

### EXAMPLE 2

Composition: 1 ml containing:

| | |
|---|---|
| Hydrogenated egg phosphatidylcholine | 36 mg |
| 1,2-Dipalmitoyl-sn-glycero-3-phosphoglycerol | 4 mg |
| sodium | |
| Sorbitol | 50 mg |
| (Trometamol | 1 mg) |
| Water for injection ad | 1 ml |

Prepared as in Example 1.

Degradation of phospholipids, measured as free fatty acids (mg/ml):

| | Without trometamol | With trometamol |
|---|---|---|
| 1 month at 30° C. | ≦1.25 | ≦1.25 |
| 1 month at 40° C. | ≦2.5 | ≦1.25 |
| 1 month at 50° C. | ≦12.5 | ≦2.5 |

The results demonstrate that the samples containing trometamol show less degradation of phospholipids, measured as free fatty acids, compared to the samples without trometamol.

Further Examples of compositions according to the present invention are prepared as in Example 1 as follows:

### EXAMPLE 3

| | |
|---|---|
| Phosphatidylcholine | 20 mg |
| Phosphatidylethanolamine | 20 mg |
| Glucose | 50 mg |
| Trometamol | 0.5 mg |
| Water for injection ad | 1 ml |

### EXAMPLE 4

| | |
|---|---|
| Phosphatidylglycerol | 5 mg |
| Sucrose | 100 mg |
| Trometamol | 1 mg |
| Water for injection ad | 1 ml |

### EXAMPLE 5

| | |
|---|---|
| PEG-phosphatidylethanolamine | 40 mg |
| Sorbitol | 50 mg |
| Trometamol | 10 mg |
| Water for injection ad | 1 ml |

### EXAMPLE 6

| | |
|---|---|
| Phosphatidylcholine | 15 mg |
| Soya oleum | 50 mg |
| Glycerol | 24 mg |
| Trometamol | 1 mg |
| Water for injection ad | 1 ml |

### EXAMPLE 7

As a further Example demonstrating the method of the present invention the following composition (as disclosed in WO-95/26205) was also tested as in Example 1.

Composition: 1 ml containing:

| | |
|---|---|
| Hydrogenated egg phosphatidylcholine (H-EPC) | 51 mg |
| Hydrogenated egg phosphatidyl serine sodium | 5 mg |
| (H-EPSNa) | |
| Iodixanol | 400 mg |
| Sorbitol | 17 mg |
| (Trometamol | 1 mg) |
| Water for injection ad | 1 ml |

The composition was prepared as in Example 1 but additionally adding an isotonic solution of iodixanol and sorbitol prior to the formation of the liposome.

Degradation of phospholipids, measured as free fatty acids (mg/ml):

| | Without trometamol | With trometamol |
|---|---|---|
| 1 month at 30° C. | ≦2.5 | ≦1.25 |
| 1 month at 40° C. | ≦5.0 | ≦2.5 |
| 1 month at 50° C. | ≦25.0 | ≦12.5 |

The results of this Example demonstrate the use of the method of the present invention in stabilising a phospholipid composition additionally containing a contrast agent.

Examples 8 to 13 below disclose the preparation of stabilized liposome suspensions suitable for use as contrast media in ultrasound (Examples 8 to 10) and magnetic resonance imaging (Examples 11 to 13) investigations. Ratios and percentages are by volume unless otherwise stated, except lipid ratios which are by weight. If ¹⁹F labelled fluorocarbons are used in Examples 8 to 10 these compositions could be used as MR contrast media.

### EXAMPLE 8

Hydrogenated egg phosphatidylcholine (HEPC) and dipalmitoylphosphate (10:1) are dissolved in chloroform-methanol (2:1), and the solvent is then removed in a rotary evaporator. The lipids are then dispersed in purified water, and the dispersion is introduced in a gas tight glass reactor equipped with a high speed emulsifier. The gas in the reactor is air with 10% C₅F₁₂. After preparation of the microbubbles, HEPES 5 mM is added.

### EXAMPLE 9

Distearoylphosphatidylcholine (DSPC), dipalmitoylphosphatidic acid (DPPA) and polyethyleneglycol (PEG 4000) in the ratio 25:1:2 are dissolved in tert-butanol, and the solvent is then removed in a rotary evaporator. The lipids are then dispersed in purified water, and the dispersion is introduced in a gas tight glass reactor equipped with a high speed emulsifier. The gas in the reactor is C₃F₈. After preparation of the microbubbles, trometamol 8 mM is added.

### EXAMPLE 10

Dipalmitoylphosphatidylcholine (DPPC), dipalmitoylphosphatidic acid (DPPA) and dipalmitoylphosphatidylethanolamine (DPPE) (8:1:1) are dissolved in chloroform-methanol-water (10:20:0.5), and the solvent is then removed in a rotary evaporator. The lipids are then dispersed in purified water, and the dispersion is introduced in a gas tight glass reactor equipped with a high speed emulsifier. The gas in the reactor is C₄H₁₀. After preparation of the microbubbles, TES 10 mM is added.

### EXAMPLE 11

Hydrogenated egg phosphatidylcholine (HEPC) and methoxy (PEG)-distearoylphosphatidylethanolamine (MPEG-DSPE) (9:1) are dry blended and dispersed in gadodiamide-caldiamide 0.5 M solution. Liposomes are then prepared by homogenisation and extrusion. HEPES 8 mM is added and the product is sterilised by autoclaving.

### EXAMPLE 12

Hydrogenated egg phosphatidylcholine (HEPC) and dipalmitoylphosphatidylglycerol (DPPG) (9:1) are dry blended and dispersed in dimegluminegadopentetate-meglumine diethylenetriaminepentetate-meglumine 0.5 M solution. Liposomes are then prepared by homogenisation and extrusion. TRIS 50 mM is added and the product is sterilised by autoclaving.

### EXAMPLE 13

Hydrogenated egg phosphatidylcholine (HEPC) is dispersed in gadodiamide 0.5 M solution. Liposomes are then prepared by homogenisation and extrusion. TES 50 mM is added and the product is sterilised by autoclaving.

### EXAMPLE 14

Composition:

| | | |
|---|---|---|
| DSPA** | 10 | mg |
| DSPG⁺⁺ | 10 | mg |
| Iohexol | 390 | mg |
| Buffer | q.s. | |
| Water for injection | ad 1 | mL |

**distearoylphosphatidic acid
⁺⁺distearoylphosphatidyl glycerol
(Both charged phospholipids)

The compositions were prepared, as in Example 1, using TRIS, HEPES or TES buffers.

TES is 1-[tris(hydroxymethyl)methyl]-2-aminoethane sulphonic acid.

The concentrations of these buffers were chosen to give identical ionic strengths in the products. The pH and degradation data are set out in full in Tables 1 and 2.

TABLE 1
Free fatty acids (mg/mL)

| | | On prep- | After 1 | After 3 |
|---|---|---|---|---|
| | On preparation | aration | month 40° C. | months 40° C. |
| Buffer | Not autoclaved | Autoclaved | Autoclaved | Autoclaved |
| TRIS 200 mM | NMT 0.5 | NMT 1.0 | NMT 5.0 | NMT 12.5 |
| target pH 7.4 | | | | |
| HEPES 168 mM | NMT 1.0 | NMT 1.0 | NMT 5.0 | NMT 12.5 |
| target pH 7.4 | | | | |
| TES 172 mM | NMT 0.5 | NMT 1.0 | NMT 5.0 | NMT 12.5 |
| target pH 7.4 | | | | |

(NMT - not more than)

TABLE 2
pH

| | | On prep- | After 1 | After 1 |
|---|---|---|---|---|
| | On preparation | aration | month 40° C. | month 50° C. |
| Buffer | Not autoclaved | Autoclaved | Autoclaved | Autoclaved |
| TRIS 200 mM | 7.49 | 7.45 | 7.36 | 7.25 |
| target pH 7.4 | | | | |
| HEPES 168 mM | 6.91 | 6.90 | 6.84 | 6.79 |
| target pH 7.4 | | | | |
| TES 172 mM | 7.22 | 7.21 | 7.15 | 7.10 |
| target pH 7.4 | | | | |

### EXAMPLE 15

Composition:

| | | |
|---|---|---|
| H-EPC* | 60 | mg |
| Iodixanol | 200 | mg |
| Sorbitol | 37 | mg |
| Buffer | q.s. | |
| Water for injection | ad 1 | mL |

*Hydrogenated egg phosphatidylcholine (a neutral phospholipid).

Three sets of compositions were prepared for comparison. In one the buffer was TRIS, HEPES or TES, in a second no buffer was used with pH being adjusted with NaOH/HCl, and in the third phosphate buffer or phosphate/citrate buffer was used. The compositions were prepared as in Example 1.

Degradation of the phospholipid, measured as free fatty acids (mg/mL) after autoclaving and after 3 months storage at 40° C. was as follows:

| Buffer | After autoclaving | 3 months, 40° C. |
|---|---|---|
| TRIS 200 mM, pH 7.4 | ≦0.25 | ≦2.5 |
| Phosphate buffer 75 mM | ≦1.0 | ≦5.0 |
| pH 7.4 | | |

(The concentrations of these buffers were chosen to give identical ionic strengths in the products).

The pH and degradation data are set out in full in Tables 3 and 4.

TABLE 3
Free fatty acids (mg/mL)

| | | On prep- | 1 month | 3 months |
|---|---|---|---|---|
| | On preparation | aration | 40° C. | 40° C. |
| Buffer | Not autoclaved | Autoclaved | Autoclaved | Autoclaved |
| TRIS 2 mM | NMT 0.25 | NMT 0.25 | NMT 1.0 | NMT 2.5 |
| target pH 7.4 | | | | |
| TRIS 8 mM | NMT 0.25 | NMT 0.25 | NMT 1.0 | NMT 2.5 |
| target pH 7.4 | | | | |
| TRIS 50 mM | NMT 0.25 | NMT 0.25 | NMT 0.5 | NMT 2.5 |
| target pH 7.4 | | | | |
| TRIS 200 mM | NNT 0.25 | NMT 0.25 | NMT 1.0 | NMT 2.5 |
| target pH 7.4 | | | | |
| TRIS 8 mM | NMT 0.25 | NMT 0.25 | NMT 0.5 | NMT 2.5 |
| target pH 8.3 | | | | |
| NaOH/HCl | NMT 0.25 | NMT 0.50 | NMT 5.0 | NMT 25 |
| target pH | | | | (˜12.5) |
| 7.4 | | | | |
| NaOH/HC1 | NMT 0.25 | NMT 0.25 | NMT 2.5 | NMT 5 |
| target pH 8.3 | | | | |
| HEPES 7 mM | NMT 0.25 | NMT 0.25 | NMT 1.0 | NMT 2.5 |
| target pH 7.4 | | | | |
| TES 7 mM | NMT 0.25 | NMT 0.25 | NMT 1.0 | NMT 5 |
| target pH 7.4 | | | | |
| Phosphate | NMT 0.25 | NMT 0.50 | NMT 1.0 | NMT 5 |
| buffer 3 mM | | | | |
| target pH 7.4 | | | | |
| Phosphate | NMT 0.25 | NMT 1.0 | NMT 5.0 | NMT 5 |
| buffer 75 mM | | | | |
| target pH 7.4 | | | | |
| Phosphate/ | NMT 0.25 | NMT 0.25 | NMT 0.5 | NMT 5 |
| citrate | | | | |
| buffer 2.6 mM | | | | |
| target pH 7.4 | | | | |

TABLE 4
pH

| | | On prep- | After 1 | After 3 |
|---|---|---|---|---|
| | On preparation | aration | month 40° C. | months 40° C. |
| Buffer | Not autoclaved | Autoclaved | Autoclaved | Autoclaved |
| TRIS 2 mM | 7.36 | 7.01 | 7.25 | 7.05 |
| target pH 7.4 | | | | |
| TRIS 8 mM | 7.34 | 7.30 | 7.28 | 7.22 |
| target pH 7.4 | | | | |
| TRIS 50 mM | 7.44 | 7.43 | 7.44 | 7.41 |
| target pH 7.4 | | | | |
| TRIS 200 mM 7.48 | 7.50 | 7.50 | 7.50 | |
| target pH 7.4 | | | | |
| TRIS 8 mM | 8.14 | 8.07 | 8.08 | 7.99 |
| target pH 8.3 | | | | |
| NaOH/HCl | 6.42 | 6.24 | 5.81 | 5.51 |
| target pH 7.4 | | | | |
| NaOH/HCl | 7.18 | 7.19 | 6.04 | 6.85 |
| target pH 8.3 | | | | |
| HEPES 7 mM | 6.99 | 6.89 | 6.83 | 6.80 |
| target pH 7.4 | | | | |
| TES 7 mM | 7.28 | 7.21 | 7.20 | 7.11 |
| target pH 7.4 | | | | |
| Phosphate | 7.46 | 7.75 | 6.71 | 6.64 |
| buffer 3 mM | | | | |
| target pH 7.4 | | | | |
| Phosphate | 7.35 | 7.17 | 7.07 | 6.99 |
| buffer 75 mM | | | | |
| target pH 7.4 | | | | |
| Phosphate/ | 7.66 | 7.74 | 6.77 | 6.65 |
| citrate | | | | |
| buffer 2.6 mM | | | | |
| target pH 7.4 | | | | |

Thus after 3 months storage, samples with TRIS and TRIS-like buffers (exemplified by HEPES and TES) show less degradation of the phospholipid as seen by the reduced level of free fatty acids compared with other buffers (phosphate buffer and phosphate/citrate buffer) and solutions without buffer (pH adjusted by NaOH/HCl). Moreover, the reduction in pH observed after autoclaving and storage was less pronounced in the samples with TRIS, HEPES and TES (a reduction of less than 0.30 pH units) compared with the other samples (a reduction of 0.30-1.00 pH units).

### EXAMPLE 16

Composition:

| | | |
|---|---|---|
| H-EPC | 36 | mg |
| MPEG-DSPE* | 4 | mg |
| Iodixanol | 370 | mg |
| Buffer | q.s. | |
| Water for injection | ad 1 | mL |

*Methoxy (polyethyleneglycol) distearoylphosphatidyl-ethanolamine (a neutral phospholipid).

Three sets of compositions were prepared for comparison. In one the buffer was TRIS, HEPES or TES, in a second no buffer was used with pH being adjusted with NaOH/HCl, and in the third phosphate buffer or phosphate/citrate buffer was used. The compositions were prepared as in Example 1.

Degradation of the phospholipid, measured as free fatty acids (mg/mL) after autoclaving and after 1 month's storage at 40° C. and 50° C. was as follows:

| Buffer | After autoclaving | 1 month 40° C. | 1 month 50° C. |
|---|---|---|---|
| TES 172 mM, | ≦0.25 | ˜0.5 | ˜1.0 |
| pH 7.4 | | | |
| Phosphate buffer | ≦1.0 | ≦2.5 | ≦5.0 |
| 74 mM, pH 7.4 | | | |
| Phosphate/citrate | ≦2.5 | ≦2.5 | ≦5.0 |
| 67 mM, pH 7.4 | | | |

The concentrations of these buffers were chosen to give identical strengths in the products.

The pH and degradation data are set out in full in Tables 5 and 6.

TABLE 5
Free fatty acids (mg/mL)

| | | On pre- | After 1 | After 1 |
|---|---|---|---|---|
| | On preparation | paration | month 40° C. | month 50° C. |
| Buffer | Not autoclaved | Autoclaved | Autoclaved | Autoclaved |
| TRIS 200 mM | NMT 0.5 | NMT 0.5 | ˜0.5 | ˜1.0 |
| target pH 7.4 | | | | |
| NaOH/HCl | NMT 0.5 | NMT 0.25 | NMT 1.25 | NMT 12.5 |
| target pH 7.4 | | | | |
| HEPES 168 mM | NMT 0.25 | NMT 0.25 | ˜0.5 | NMT 2.5 |
| target pH 7.4 | | | | |
| TES 172 mM | NMT 0.25 | NMT 0.25 | ˜0.5 | ˜1.0 |
| target pH 7.4 | | | | |
| Phosphate | NMT 0.5 | NMT 1.0 | NMT 2.5 | NMT 5.0 |
| buffer 74 mM | | | | |
| target pH 7.4 | | | | |
| Phosphate/ | NMT 0.25 | NMT 2.5 | NNT 2.5 | NMT 5.0 |
| citrate | | | | |
| buffer 67 mM | | | | |
| target pH 7.4 | | | | |

TABLE 6
pH

| | | | After 1 | After 1 |
|---|---|---|---|---|
| | On prepara- | On pre- | month | month |
| | tion Not | paration | 40° C. | 50° C. |
| Buffer | autoclaved | Autoclaved | Autoclaved | Autoclaved |
| TRIS 200 mM | 7.43 | 7.44 | 7.42 | 7.40 |
| target pH 7.4 | | | | |
| NaOH/HCl | 6.52 | 5.84 | 5.54 | 4.98 |
| target pH 7.4 | | | | |
| HEPES | 6.91 | 6.89 | 6.89 | 6.91 |
| 168 mM | | | | |
| target pH 7.4 | | | | |
| TES 172 mM | 7.18 | 7.16 | 7.18 | 7.19 |
| target pH 7.4 | | | | |
| Phosphate | 7.37 | 7.20 | 7.23 | 7.22 |
| buffer 74 mM | | | | |
| target pH 7.4 | | | | |
| Phosphate/ | 7.52 | 7.04 | 7.25 | 7.24 |
| citrate | | | | |
| buffer 67 mM | | | | |
| target pH 7.4 | | | | |

1. A method for stabilising a substantially saturated phospholipid composition, which method comprises including in a substantially saturated phospholipid composition a buffer system comprising TRIS, BIS-TRIS, TES or HEPES having a pH at 15° C. of less than or equal to 9.5 and autoclaving the buffer system containing composition, wherein said buffer system containing composition comprises fully formed liposomes prior to autoclaving, the inclusion of said buffer system being other than by adding a said buffer system to a liposomal composition containing a nonionic multiply hydroxylated X-ray contrast agent having said agent present within the liposomes and within the surrounding aqueous medium at substantially the same concentration.

2. A method as claimed in claim 1 wherein said buffer system is added to a pre-formed liposomal composition.

3. A method as claimed in claim 1 wherein said buffer system is admixed with a phospholipid composition and liposomes are generated in the resulting phospholipid and buffer containing mixture.

4. A method as claimed in claim 1 wherein said composition contains a contrast effective material.

5. A method as claimed in claim 4 wherein said contrast effective material is an echogenic material.

6. A method as claimed in claim 4 wherein said contrast effective material is a paramagnetic material.

7. A method as claimed in claim 4 wherein said contrast effective material is a radiation emitting material.

8. A method as claimed in claim 4 wherein said contrast effective material is an iodinated organic compound.

9. A method as claimed in claim 4 wherein said contrast effective material is present within liposomes in said composition.

10. A method as claimed in claim 1 wherein said composition contains a therapeutic or prophylactic agent.

11. A method as claimed in claim 1 wherein said composition contains a cosmetic agent.

12. A method as claimed in claim 1 wherein said phospholipids are neutral phospholipids.

13. A method of contrast enhanced imaging in which a contrast medium is administered to a subject and an image of the subject is generated, characterised in that as said contrast medium is used an aqueous lipid composition containing a contrast effective material, said composition comprising one or more substantially saturated phospholipids in combination with a buffer system comprising TES, TRIS, BIS-TRIS or HEPES having a pH at 15° C. of less than or equal to 9.5, said composition having been autoclaved while containing said buffer system and fully formed liposomes, with the proviso that where said phospholipids comprise a combination of charged and neutral phospholipids and said composition is a liposome composition containing a nonionic multiply hydroxylated X-ray contrast agent, then said agent is not present within the liposomes and within the surrounding aqueous medium at substantially the same concentration.

14. A method as claimed in claim 16 wherein image generation is effected by X-ray, ultrasound, MR imaging or nuclear medicine imaging.
